(19) United States
(12) Patent Application Publication
(10) Pub. No.: US 2009/0170070 A1
(43) Pub. Date: Jul. 2, 2009

(54) INCREASED SPECIFICITY OF ANALYTE DETECTION BY MEASUREMENT OF BOUND AND UNBOUND LABELS

(75) Inventors: Sieglinde Neerken, Eindhoven (NL); Gerhardus Wilhelmus Lucassen, Eindhoven (NL); Kristiane Anne Schmidt, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/304,564

(22) PCT Filed: Jun. 7, 2007
(86) PCT No.: PCT/IB07/52159

§ 371 Date: Dec. 12, 2008

(30) Foreign Application Priority Data
Jun. 15, 2006 (EP) 06115558.6

(51) Int. Cl. C12Q 1/70; C12Q 1/68

(52) U.S. Cl. 435/5; 435/6

(57) ABSTRACT

The present invention describes the provision of an internal control in analytical techniques involving labeling of analytes, such as SERRS, for detection of an analyte, particularly a biomolecule in a sample, with improved accuracy.

### FIELD OF THE INVENTION

The present invention relates to a method for the detection of an analyte, particularly a biomolecule in a sample, by an analytical technique involving labeling of analytes, wherein the accuracy and/or reliability of the measurement is of importance.

### BACKGROUND OF THE INVENTION

The sensitive and accurate detection, either qualitatively or quantitatively, of low concentrations of biomolecules such as proteins, peptides, oligonucleotides, nucleic acids, lipids, polysaccharides, hormones, neurotransmitters, metabolites, etc. has proven to be an elusive goal with widespread potential uses in medical diagnostics, pathology, toxicology, epidemiology, biological warfare, environmental sampling, forensics and numerous other fields.

A particular example is the detection of DNA e.g. in medical diagnostics (the detection of infectious agents like pathogenic bacteria and viruses, the diagnosis of inherited and acquired genetic diseases, etc.), in forensic tests as part of criminal investigations, in paternity disputes, in whole genome sequencing, etc.

While the identification and/or quantification of a purified sample can sometimes be performed based on the physicochemical properties of the analyte itself, many detection assays make use of a ‘probe’, which is a known molecule having a strong affinity and preferably also a high degree of specificity for the analyte. Where the analyte is a protein or peptide, these assays are referred to as ligand-binding assays. One of the most common ligand-binding assays are immunoassays. Immunoassays typically employ an antibody which specifically binds to the antigen within the analyte to form an antibody-antigen complex. Ligand binding assays are especially relevant to medical diagnostics. In modern medical practice, ligand binding assays are routinely run on patients' blood, urine, saliva, etc. in order to determine the presence or levels of antibodies, antigens, hormones, medications, poisons, toxins, illegal drugs, etc. Ligand binding assays are also being used to monitor groundwater contamination, toxins and pesticides in foods, industrial biological processes, and in many areas of biological research.

Detection of DNA typically makes use of the hybridization of a ‘probe’, which is a nucleotide sequence specific for the target DNA. Such assays are commonly used in the specific detection of active infectious agents, for the identification of DNA in forensic analysis and in the identification of genetic defects.

A common feature in these detection assays is often the labeling of analyte-specific probe with a traceable substance. The detection of the traceable substance (hereafter referred to as label), bound to the analyte, is indicative of the amount of analyte in the sample. Detection of the label can be ensured using one of a variety of different techniques depending upon the nature of the label employed. Current detection methods typically involve detection of fluorescently labeled antibodies or oligonucleotide probes that can bind to the analyte or target biomolecule. Cross-reactivity and non-specific binding may complicate fluorescent detection of biomolecules in complex samples. Even where high probe-specificity is obtained, the sensitivity of fluorescent detection is often insufficient to identify low concentrations of biomolecules. This is particularly true when the biomolecule to be detected is present at low concentrations in a complex mixture of other molecules, where interference, fluorescence quenching and high background fluorescence may all act to obscure or diminish the signal from the target biomolecule.

Surface enhanced (resonance) Raman spectroscopy or SE(R)RS is a technique that is rapidly gaining on fluorescence for detection in view of its impressive sensitivity. Raman spectroscopy utilizes the phenomenon of Raman scattering. When light passes through an optically transparent sample, a fraction of the light is scattered in all directions. Most of the scattered photons are of the same wavelength as the incident light. This is known as Rayleigh scattering. However, a small fraction of the scattered light has a different wavelength and a slight random alteration in phase. The wavelengths of the Stokes (Anti-Stokes) Raman emission spectrum are shifted to longer (or shorter) wavelengths relative to the excitation wavelength. The Raman spectrum is characteristic of the chemical composition and structure of the light absorbing molecules in a sample, while the intensity of Raman scattering is dependent on the concentration of these molecules. The intrinsically weak Raman scattering can be enhanced by factors of up to 108 or more when a compound is adsorbed on or near roughened metal surfaces, e.g. nanoparticles of gold, silver, copper and certain other metals. The technique associated with this phenomenon is known as surface-enhanced Raman scattering (SERS). The increase in detection sensitivity is more marked the closer the analyte sits to the “active” surface. The optimum position is in the first molecular layer around the surface, i.e. within about 30 nm of the surface. This can be achieved by for example spermine that neutralizes the net charge on nucleic acids so that the molecules can be in close proximity to the silver surface.

A further 10³ to 10⁵-fold increase in sensitivity can be obtained by operating at the resonance frequency of the analyte or, as is more commonly done, making use of a ‘SERS-active’ substance or dye attached to the analyte (capable of generating a SE(R)RS spectrum when appropriately illuminated), and operating at the resonance frequency of the dye. This is termed “resonance Raman scattering” spectroscopy. The combination of the surface enhancement effect and the resonance effect to give “surface enhanced resonance Raman scattering” or SERRS strongly increases the sensitivity. Compared to fluorescence a SERRS signal can be more easily discriminated from contamination and background.

Another key advantage of SE(R)RS is the possibility of multiplexing using a single excitation wavelength. Each SE(R)RS-active dye used as a label gives a unique fingerprint which can be recognized in a dye mixture without separation as would be necessary for fluorescence spectroscopy. There are about 50 specially designed dyes for SE(R)RS, each of which gives a unique spectrum. SE(R)RS is thus a highly sensitive and specific method for biomolecule detection giving sufficient sensitivity to detect low concentrations of biomolecules. Bioanalytical techniques using SE(R)RS have been demonstrated to allow detection of attomole (10⁻¹⁸ mol) quantities of proteins or DNAs down to femtomole (10⁻¹⁵ mol in 400 μl) concentrations. Single-molecule detection limits have been reported for rhodamine 6G, adenine, crystal violet, and other SERRS-active molecules. Raman spectroscopy is applied very broadly, from material analysis in physics to a very wide variety of applications in biology.

Even though very sensitive detection methods such as SE(R)RS are available, accurate quantitative analysis remains a challenge. Each method typically requires the production of reagents and the provision of specific detection conditions, the variability of which can affect accuracy of the detection.

Also for SE(R)RS based detection methods, various factors have been reported to affect the reliability and accuracy of detection. SE(R)RS-active surfaces have a complex structure and dynamics which makes it difficult to manufacture them in a reproducible manner. Moreover, the SE(R)RS enhancement is strongly dependent on the distance between the analyte and the SE(R)RS-active surface. Furthermore, variations of SE(R)RS enhancement occur with the surface coverage of the analyte on the SE(R)RS-active surface (related to the distribution of SE(R)RS-active hot spots). In addition, quantitative concentration measurements using optical methods (including SE(R)RS as well as normal Raman or fluorescence) must contend with intensity variations produced by changes in excitation, collection efficiency, or both.

In the art, correcting for such variations is most often approached using an internal and/or external standard to calibrate the correlation between the optical signal and the concentration (or amount) for the analyte of interest. It has been proposed to improve the accuracy for SE(R)RS (colloidal) quantification by using the SE(R)RS signal generated from a self-assembled monolayer (SAM) as an internal standard. With this method, the high coverage of the SAM is presumed to prevent chemisorption of the analyte onto the SE(R)RS-active surfaces and thus to improve reproducibility. However, this approach was found to have a number of intrinsic limitations resulting in relatively large prediction errors (root mean prediction error of 0.5 M for samples between 0.1 and 5 M) observed when using this SAM internal standard method.

It has been proposed that by ensuring that the analyte and internal standard molecules have virtually identical chemical properties, their relative SE(R)RS intensity is far less sensitive to batch-to-batch colloid solution variations and optical excitation/collection parameters. This would allow for improvement of the accuracy of quantitative SE(R)RS measurements over a wide concentration range with improved accuracy and reproducibility.

### SUMMARY OF THE INVENTION

The object of the invention is to provide an alternative or improved method for the detection, e.g. qualitatively or quantitatively, of an analyte by a detection technique involving labeling of the analyte. An advantage of the method is improved reliability and/or accuracy of the measurement.

In an aspect of the present invention, an internal reference is included to thereby ensure improved reliability and accuracy of detection. More particularly, this is achieved by introducing an additional measurement that is equally determined by the presence and/or amount of analyte in the sample and thus can serve as an internal reference for the direct detection of the analyte. When working with a predetermined amount of label, the detection and/or quantification of the fraction of unbound label provides an internal reference on the direct detection/quantification of the analyte which is based on the measurement of the fraction of label bound thereto.

The present invention thus provides methods for detecting and optionally quantifying the presence of an analyte in a sample, comprising the steps of: a) contacting the sample potentially comprising the analyte with a predetermined amount of label capable of binding to the analyte; b) detecting the fraction of label bound to the analyte, whereby the amount of label bound to the analyte is indicative of the presence (and optionally of the amount) of analyte in the sample; and further comprising the step of (c) detecting the fraction of label not bound to the analyte, whereby the amount of label not bound to the analyte provides an internal control indirectly indicative of the presence (and optionally the amount of analyte in the sample) wherein said detection step in (b) and (c) is ensured using an optical detection method.

According to one embodiment, the detection of the fraction of label bound to the analyte and the detection of the fraction of label, which is not bound to the analyte, is performed without prior separation, i.e. within the same sample. This can be achieved e.g. by the use of a label, which can be differentially detected in a bound or unbound state. The labels that are used are optical labels. According to a particular embodiment, use is made of a label which is a fluorescent and/or a SE(R)RS-active label of which the maximum absorption frequency is shifted from a first to a second frequency on association of said fluorescent and/or SE(R)RS-active label with said SE(R)RS-active surface. Alternatively, use is made of a label, which is provided as a molecular beacon, so as to ensure a different signal for the label when bound to the analyte and when not bound to the analyte.

According to an alternative embodiment, the methods of the present invention further include, prior to step (b), a separation step, whereby the fraction of label bound to the analyte is separated from the fraction of label not bound to the analyte. This separation step can involve the removal of one or both fractions from the sample or can be a physical separation of the fractions within one sample.

According to one embodiment this is achieved by capturing the fraction of bound label on a substrate and physically removing the substrate or the fraction of unbound label from the sample. The fraction of bound label can be captured on a substrate by way of a capture probe, which captures the analyte on the substrate. According to one embodiment, the capture probe is an analyte-specific capture probe. Alternatively, the binding of the analyte to the substrate occurs through a biotin-tag on the analyte, which is contacted with a streptavidin tag on the substrate. The biotin tag can be incorporated into the analyte by PCR amplification.

Further specific embodiments of the present invention relate to methods wherein the separation of the bound and unbound fraction of label is ensured by the binding of the bound fraction to a substrate or a tag which is reactive to a physical or chemical force, and application of the physical or chemical force (such as a gravitational force, magnetic field) is used to (re)move the bound fraction.

The methods of the present invention are applicable to the detection of virtually any type of analyte, such as, but not limited to a nucleic acid, a protein, a carbohydrate, a lipid, a chemical substance, an antibody, a microorganism, or a eukaryotic cell. Particular embodiments of the methods of the present invention relate to the detection and/or quantification of nucleic acid sequences such as DNA.

Most particularly, the methods of the present invention are methods whereby the detection steps (b) and (c) described above are ensured using an optical detection method. Most specifically, the detection steps (b) and (c) of the methods of the present invention are ensured using SE(R)RS, whereby the label is a SE(R)RS-active label. In a particular embodiment, the methods thus further comprise, prior to step (b) and (c), a step which involves the contacting of the fraction of label bound to the analyte and of the fraction of label not bound to the analyte with a SE(R)RS-active surface. Where the detection of the bound and unbound label is performed within the same sample, the bound and unbound label are contacted with the SE(R)RS-active surface simultaneously, by adding the SE(R)RS-active surface to the sample. In a particular embodiment the SE(R)RS-active surface used in the methods of the present invention is a colloidal suspension of silver or gold nanoparticles, or aggregated colloids thereof.

Typically, when working with a sample in which the analyte is not present in a purified form, i.e. a sample wherein other components are present, the label used in the methods of the present invention is an analyte-specific label, i.e. capable of binding specifically to the analyte. Nevertheless it is also envisaged that where the analyte is present in a purified form, and the methods of the invention are used for quantification purposes, it is sufficient that the label binds to the analyte.

Where the label used in the methods of the present invention is an analyte-specific label, this can be ensured by using an analyte-specific probe bound to a label. Typically, where the analyte is a nucleotide sequence, the analyte-specific probe can be a complementary oligonucleotide sequence.

The present invention also provides a system for detecting and/or quantifying the presence of an analyte in a sample, comprising:

a) means for contacting said sample potentially comprising said analyte with a predetermined amount of label capable of binding to said analyte
b) means for detecting the fraction of label bound to said analyte; whereby the amount of label bound to said analyte is indicative of the presence and optionally of the amount of analyte in said sample; and
c) means for detecting the fraction of label not bound to said analyte; whereby the amount of label not bound to said analyte, deducted from said predetermined amount of label, provides an internal control indicative of the presence and optionally the amount of analyte in said sample.

The system may be used for analysis of analyte, e.g. in molecular diagnosis.

The above and other characteristics, features and advantages of the present invention will become apparent from the following detailed description, taken in conjunction with the accompanying drawings, which illustrate, by way of example, the principles of the invention. This description is given for the sake of example only, without limiting the scope of the invention. The reference figures quoted below refer to the attached drawings.

### BRIEF DESCRIPTION OF THE DRAWINGS

FIG. 1 is a schematic drawing of a particular embodiment of the detection method of the present invention to measure analyte-bound and unbound label.

FIG. 2 is a schematic drawing of an embodiment of the method of the present invention as applied to SE(R)RS detection of DNA in a sample.

FIG. 3A is an example of SE(R)RS spectra of analyte-bound and unbound labels, according to one embodiment of the invention. A comparison of the spectra gives extra information on the concentration of the analyte.

FIG. 3B is an example of the fluorescence spectra of spectra of analyte-bound and unbound labels, according to one embodiment of the invention. A comparison of the spectra gives extra information on the concentration of the analyte.

FIGS. 4A and B are schematic representations of systems according to different embodiments of the present invention.

### DETAILED DESCRIPTION OF THE INVENTION

The term “analyte”, as used herein, refers to the substance to be detected in the test sample using the present invention. A non-limiting list of analytes envisaged by the present invention is provided in the description.

The term “label”, as used herein, refers to a molecule or material capable of generating a detectable signal. A non-limiting list of labels envisaged for use in the methods of the present invention is provided in the description.

The term “fraction of bound label” as used herein refers to those labels, which, when adding a predetermined amount of label to a sample, bind to the analyte.

The term “fraction of unbound label” as used herein refers to those labels which, when adding a predetermined amount of label to a sample, do not bind to the analyte.

It will be understood that, in the methods of the present invention, reference is made to the fractions of bound and unbound label, independently of whether, upon detection, any label is detected in the relevant fraction.

An “analyte-specific probe” as used herein, is a probe capable of specifically binding to the analyte and to which a label can be attached. The binding of the probe to the analyte can be based on any type of interaction including but not limited to complementary nucleotide sequences, antigen/antibody interaction, ligand/receptor binding, enzyme/substrate interaction, etc.

An “analyte-specific label” as used herein, refers to a label, which is capable of specifically binding to the analyte, either by its inherent characteristics or as a result of the label being linked to an analyte-specific probe.

A “capture probe” as used herein refers to a molecule capable of binding a molecule or a complex of molecules to a substrate.

A “substrate” as used herein refers to a material, to which molecules or complexes of molecules can be bound, and which can be manipulated. Typical examples of substrates include but are not limited to microtiter plates, beads, chips, etc.

A “SE(R)RS-active surface” as used herein refers to a metal surface that contributes to strong enhancement of Raman scattering when analytes are adsorbed or in close proximity to it. The surface may e.g. be an etched or roughened metallic surface, a metal sol, or an aggregation of metal colloid particles. A more extensive list of SE(R)RS-active surfaces is to be found in the description below.

The present invention will be described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated.

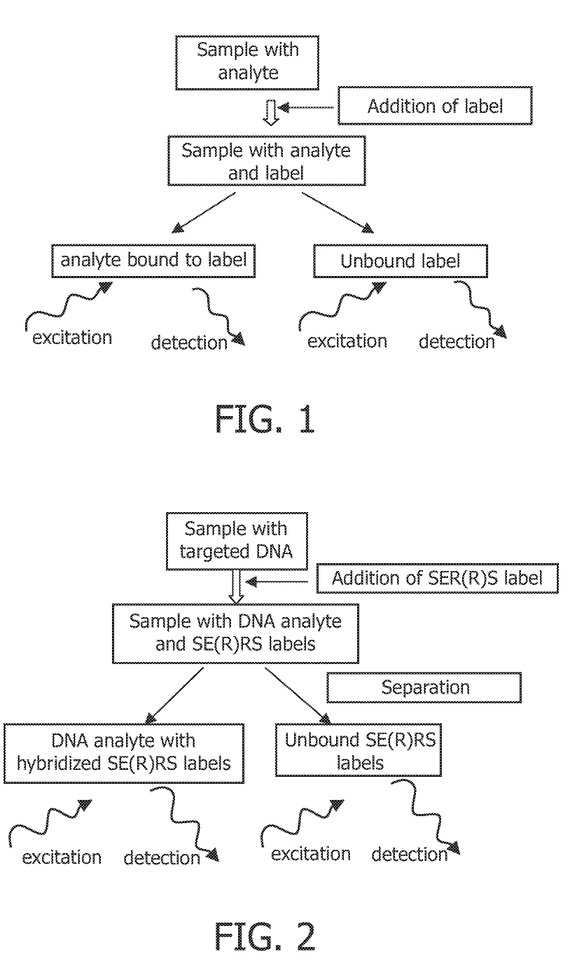

According to a first aspect, the present invention provides an analytical technique for the detection and/or quantification of an analyte in a sample based on the labeling of the analyte, whereby a predetermined amount of label is contacted with the sample and, in addition to the detection and/or quantification of the bound label, also the unbound fraction of the label is quantified. Thus, according to one embodiment, the method of the invention includes the steps of (FIG. 1):

contacting a sample suspected to contain an analyte with a predetermined amount of label capable of binding to the analyte, and

detecting and/or quantifying the fraction of label bound to the analyte (bound fraction of label) and

detecting and/or quantifying the fraction of label not bound to the analyte (unbound fraction of label).

The invention is based on the concept that, when working with a predetermined amount of label (total label or 100%), detection of the bound fraction of label provides direct information on the presence and/or concentration of the analyte (x or a % of total label), while at the same time detection of the unbound fraction of label (y or b % of total label) should indirectly provide the same information (total label−y=x; or 100%−b %=a %) and can thus serve as an internal reference or control.

The introduction of an internal reference according to the method of the present invention ensures a more accurate and reliable detection of analytes.

The method of the present invention can in principle be applied to any analytical detection technique whereby detection is based on the binding of a label to the analyte and detection of the analyte-bound label. Most particularly, the methods of the present invention are suitable for detection methods, which allow the accurate quantitative detection of label over a wide range of concentrations. Typically, detection methods based on detection using a label require the addition of an excess of label to the sample to ensure accurate detection. Depending on the amount of analyte in the sample, the fraction of unbound label varies from very large (i.e. close to or the same as the predetermined amount of excess label added) to very small. Typically, when working with DNA probes, concentrations of label within a range of 10⁻⁶ M and 10⁻⁹ M are used.

The methods of the present invention are methods, which involve the detection of an analyte. The nature of the analyte to be detected is not critical to the invention and can be any molecule or aggregate of molecules of interest for detection. A non-exhaustive list of analytes includes a protein, polypeptide, peptide, amino acid, nucleic acid, oligonucleotide, nucleotide, nucleoside, carbohydrate, polysaccharide, lipopolysaccharide, glycoprotein, lipoprotein, nucleoproteins, lipid, hormone, steroid, growth factor, cytokine, neurotransmitter, receptor, enzyme, antigen, allergen, antibody, metabolite, cofactor, nutrient, toxin, poison, drug, biowarfare agent, biohazardous agent, infectious agent, prion, vitamin, immunoglobulins, albumin, hemoglobin, coagulation factor, interleukin, interferon, cytokine, a peptide comprising a tumor-specific epitope and an antibody to any of the above substances. An analyte may comprise one or more complex aggregates such as but not limited to a virus, bacterium, fungus, microorganism such as Salmonella, Streptococcus, Legionella, E. coli, Giardia, Cryptosporidium, Rickettsia, spore, mold, yeast, algae, amoebae, dinoflagellate, unicellular organism, pathogen or cell, and cell-surface molecules, fragments, portions, components, products, small organic molecules, nucleic acids and oligonucleotides, and metabolites of microorganisms.

According to a particular embodiment, an analyte is a DNA such as a gene, viral DNA, bacterial DNA, fungal DNA, mammalian DNA, or DNA fragments. The analyte can also be RNA such as viral RNA, mRNA, rRNA. The analyte can also be cDNA, oligonucleotides, or synthetic DNA, RNA, PNA, synthetic oligonucleotides, modified oligonucleotides or other nucleic acid analogue. It may comprise single-stranded and double-stranded nucleic acids. It may, prior to detection, be subjected to manipulations such as digestion with restriction enzymes, copying by means of nucleic acid polymerases, shearing or sonication thus allowing fragmentation to occur.

The invention is particularly suited for detection methods, which involve detection by use of a label, such as, but not limited to, a fluorescent, chromogenic or chemiluminescent dye, a radio-isotope, metal and/or magnetic nanoparticle, etc.

Accordingly, the detection steps performed in the methods of the invention will be determined by the label used and include, but are not limited to fluorescence, colorimetry, absorption, reflection, polarization, refraction, electrochemistry, chemiluminescence, Rayleigh scattering and Raman scattering, SE(R)RS, resonance light scattering, grating-coupled surface plasmon resonance, scintillation counting, magnetic sensors, electrochemical detection (such as anode stripping voltametry), etc.

Suitable labels for use in the different detection methods are numerous and extensively described in the art. Fluorescent labels include but are not limited to fluorescein isothiocyanates (FITC), carboxyfluoresceins, such as tetramethylrhodamine (TMR), carboxy tetramethyl-rhodamine (TAMRA), carboxy-X-rhodamine (ROX), sulforhodamine 101 (Texas Red™), Atto dyes (Sigma Aldrich), Fluorescent Red and Fluorescent Orange, phycoerythrin, phycocyanin, and Crypto-Fluor™ dyes. The most common radioisotopes include beta-emitters such as ³H and ¹⁴C, and gamma-emitters, such as iodine-125 (¹²⁵I). Other described labels used in quantitative and qualitative assays include but are not limited to dendrimers, quantum dots, up-converting phosphors and nanoparticles.

The method of the present invention is particularly suitable for detection methods based on surface-enhanced (resonance) Raman spectroscopy (SE(R)RS), which allows for sensitive quantitative detection in a wide range of concentrations.

Where the detection of the analyte in the methods of the invention is based on SE(R)RS, the label is a material which is SE(R)RS-active, i.e. which is capable of generating a SERS or SERRS spectrum when appropriately illuminated, also referred to herein as a SER(R)S-active label or dye. Non-limiting examples of SE(R)RS-active labels include fluorescein dyes, such as 5-(and 6-)carboxy-4′,5′-dichloro-2′,7′-dimethoxy fluorescein, 5-carboxy-2′,4′,5′,7′-tetrachlorofluorescein and 5-carboxyfluorescein; rhodamine dyes such as 5-(and 6-)carboxy rhodamine, 6-carboxytetramethyl rhodamine and 6-carboxyrhodamine X, phthalocyanines such as methyl, nitrosyl, sulphonyl and amino phthalocyanines, azo dyes such as those listed in U.S. Pat. No. 6,127,120, azomethines, cyanines and xanthines such as the methyl, nitro, sulphano and amino derivatives, and succinylfluoresceins. Each of these may be substituted in any conventional manner, giving rise to a large number of useful labels.

According to a particular embodiment the SE(R)RS-active label is a carboxy rhodamine, FAM or TET. It has been demonstrated that a calibration curve for an oligonucleotide labeled with carboxyrhodamine R6G reaches a detection limit of 1.05×10⁻¹² M (which, taking into account dilution effects, corresponded to a detection of 0.5 femtomoles of the labeled oligonucleotide in the sample volume). At the same time, the calibration graph of R6G (as well as for FAM and TET) has been shown to be linear over a range from 10⁻⁷ M to 10⁻¹¹ M (LGC ‘Evaluation of the sensitivity of SERRS-based DNA detection, January 2004, LGC/Mfb/2004/02, available at—http://www.mfbprog.org.uk/themes/theme_publications_item.asp?intThemeID=10&intPublicationID=865).

It is noted that the choice of the label can be influenced by factors such as the resonance frequency of the label, the resonance frequency of other molecules present in the sample, etc. SE(R)RS-active labels of use for detecting biomolecules are described in the art such as in U.S. Pat. No. 5,306,403, U.S. Pat. No. 6,002,471, and U.S. Pat. No. 6,174,677.

Detection by surface-enhanced spectroscopies such as surface-enhanced (resonance) Raman spectroscopy (SE(R)RS) is based on the strong enhancement of Raman scattering observed for analytes adsorbed onto a roughened metal surface which can be colloids. Thus, this requires the detection of the label in the presence of an appropriate ‘SE(R)RS-active surface’. Typically, the surface is a noble (Au, Ag, Cu) or alkali (Li, Na, K) metal surface. The metal surface may for instance be an etched or otherwise roughened metallic surface, a metal sol or, according to a particular embodiment, an aggregation of metal colloid particles as the latter results in enhancements for SERRS of greater than 10⁸-10¹² of the Raman scattering. The metal nanoparticles making up the SE(R)RS-active surface in the detection methods of the present invention can also be arranged in metal nanoparticle island films, metal-coated nanoparticle-based substrates, polymer films with embedded metal nanoparticles, and the like. The metal surface may be a naked metal or may comprise a metal oxide layer on a metal surface. It may include an organic coating such as of citrate or of a suitable polymer, such as polylysine or polyphenol, to increase its sorptive capacity.

According to a particular embodiment of the invention, the metal colloid particles making up the SE(R)RS-active surface are nanoparticles or colloidal nanoparticles aggregated in a controlled manner such as described in US 20050130163 A1. Alternative methods of preparing nanoparticles are known (e.g. U.S. Pat. Nos. 6,054,495, 6,127,120, 6,149,868). Nanoparticles may also be obtained from commercial sources (e.g. Nanoprobes Inc., Yaphank, N.Y.; Polysciences, Inc., Warrington, Pa.). The metal particles can be of any size so long as they give rise to a SE(R)RS effect. Typically they have a diameter of about 4-50 nm, most particularly between 25-40 nm, depending on the type of metal.

In the detection and/or quantification methods of the present invention making use of SE(R)RS detection methods, it is envisaged that (at least) one of the components used or a combination thereof, i.e. the label, the probe, the labeled probe, the analyte or the label-bound analyte is adsorbed onto a metal surface. Such adsorption can be mediated by direct binding or by a linker compound, involving either non-covalent or covalent attachment. Various options and modes of adsorption are known in the art and described e.g. in U.S. Pat. Nos. 6,127,120 and 6,972,173. Typically adsorption to the metal SE(R)RS-active surface is ensured by addition of a monomeric or polymeric polyamine, more particularly a short-chain aliphatic polyamine, such as spermine. Thus, according to one embodiment, the methods of the invention will comprise, prior to detection, addition of a polyamine to the sample to be detected by SE(R)RS.

Alternatively or additionally, the analyte-specific probe is modified so as to promote or facilitate chemi-sorption onto the SE(R)RS-active surface. This can be ensured by at least partially reducing the overall negative charge of the analyte-specific probe. More particularly, where the analyte-specific probe is a nucleotide, this can be ensured by incorporating into the nucleic acid or nucleic acid unit one or more functional groups comprising a Lewis base, such as amino groups, as described in U.S. Pat. No. 6,127,120.

According to a further embodiment, a functional group (such as e.g. a Lewis base) is provided on the SE(R)RS-active label so as to promote or facilitate chemi-sorption onto the SE(R)RS-active surface. Optionally, the SE(R)RS-active label or dye and metal particles are entrapped in a polymer bead as described in US 2005/0130163, which can optionally further contain magnetic particles, rendering the beads magnetic which can be of interest in separation (see below).

Where one or more of the label, the probe, or the labeled probe is/are adsorbed to the SE(R)RS-active surface, detection of both bound and unbound label can be ensured in a similar way.

According to an alternative embodiment of the invention, the analyte is adsorbed to the metal SE(R)RS-active surface, e.g. by use of the chemical modifications described above or way of a specific linker. According to this embodiment, the unbound label, when separated from the bound label, is not in contact with the SE(R)RS-active surface. In order to ensure detection of the unbound label, it can be contacted with a metal SE(R)RS-active surface. Optionally this can be ensured by contacting the probe with a metal SE(R)RS-active surface (or an excess of analyte which has been bound to a metal SE(R)RS-active surface).

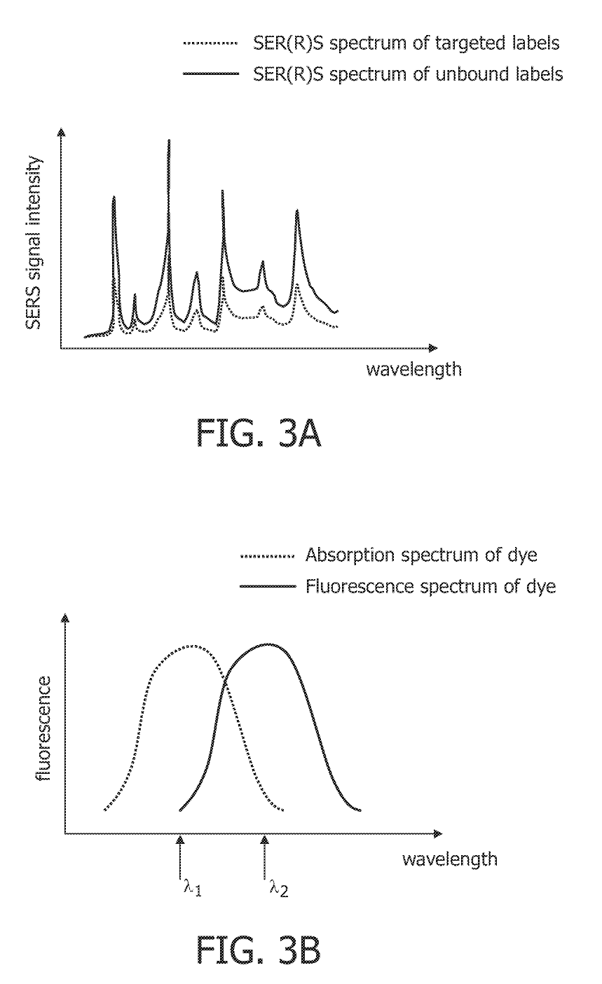

The methods of the present invention involve the detection of both the fraction of label bound to the analyte (fraction of bound label) as the fraction of label not bound to the analyte (fraction of unbound label). According to one embodiment of the invention, the fraction of bound and unbound label are measured using the same detection method. Thus, according to this embodiment, both the bound and unbound fraction are detected using e.g. SE(R)RS or fluorescence (FIGS. 3a and 3b). Alternatively, however it is envisaged that the bound and unbound fraction can be measured using different detection methods. According to the latter embodiment the bound label can be measured e.g. using SE(R)RS, while the unbound label can be measured based on another detection method e.g. fluorescence. It will be understood that this requires the use of a label, which is detectable in two different methods or the use of a double label. As the SE(R)RS spectrum of a dye is molecule-specific, most fluorescent dyes can in principle also be detected based on their SE(R)RS spectrum. The use of different detection methods for the detection of the bound and unbound fraction of the label in the methods of the present invention is of interest where e.g. the SE(R)RS-active surface is bound to the analyte (see above).

As indicated above, the method of the present invention can be applied in any method which involves detection of an analyte by binding to a label. While binding of the label to the analyte is critical, it is envisaged that this binding needs not necessarily be analyte-specific. Where the method of the invention is applied for the quantitative detection of pure analyte, it is indeed sufficient that the label is capable of binding to the analyte (as long as binding does not occur with any of the materials used in the assay, e.g. the sample container). The ability of a label to bind to an analyte can be based on inherent binding of the label to the analyte, e.g. random incorporation of a dye in between double stranded DNA.

Typically, however, where detection of an analyte in a sample is required, the binding of the label to the analyte should be a specific binding by using an analyte-specific label. According to one embodiment this is ensured by linking a label to an analyte-specific “probe”. The nature of the analyte-specific probe will be determined by the nature of the analyte to be detected. Most commonly, the probe is developed based on a specific interaction with the analyte such as, but not limited to antigen-antibody binding, complementary nucleotide sequences, carbohydrate-lectin, complementary peptide sequences, ligand-receptor, coenzyme-enzyme, enzyme inhibitors-enzyme etc. The analyte-specific probe linked to the label results in an “analyte-specific label”, which, according to this embodiment of the invention, is a label capable of binding specifically to the analyte.

According to a particular embodiment of the present invention, the analyte of interest is a oligonucleotide and the analyte-specific probe is a oligonucleotide probe, of which the sequence is complementary to the analyte of interest. This oligonucleotide probe is bound to a label so as to obtain an analyte-specific label.

Methods for preparing labeled nucleotides and incorporating them into nucleic acids are described in the art (e.g. U.S. Pat. No. 4,962,037; U.S. Pat. No. 5,405,747; U.S. Pat. No. 6,136,543; U.S. Pat. No. 6,210,896).

In a particular embodiment of the invention, a SE(R)RS-active label is used, which is either attached directly to the oligonucleotide probe or via a linker compound. SE(R)RS-active labels that contain reactive groups designed to covalently react with other molecules, such as nucleotides or nucleic acids, are commercially available (e.g., Molecular Probes, Eugene, Oreg.). SE(R)RS-active labels that are covalently attached to nucleotide precursors may be purchased from standard commercial sources (e.g., Roche Molecular Biochemicals, Indianapolis, Ind.; Promega Corp., Madison, Wis.; Ambion, Inc., Austin, Tex.; Amersham Pharmacia Biotech, Piscataway, N.J.).

Where the analyte is a nucleotide sequence, the analyte-specific label can either be a probe which can be used in the specific detection of the analyte by hybridising of the analyte-specific probe to the analyte and detection of the bound and unbound label according to the invention. Alternatively, the methods of the invention can involve amplification of the analyte using e.g. PCR, whereby the analyte-specific label is incorporated into the PCR product. According to the methods of the present invention, a predetermined amount of analyte-specific label (or labeled primer) is used and both the incorporated analyte-specific label and the (amount of) unbound analyte-specific label is detected.

The present invention relates to a method of detection and/or quantification of an analyte based on binding of a label to the analyte whereby accuracy and reliability of detection is improved by contacting the sample with a predetermined amount of label and detection of both the bound fraction and the unbound fraction of the label.

According to one embodiment of the invention, the bound and unbound fraction of the label can be individually detected and/or quantified without prior separation, i.e. within the same sample. This can be achieved according to one embodiment of the invention by use of an (analyte-specific) label of which the signal is modified upon binding to the analyte. An example of such a label are labels bound to a molecular beacon. For instance, use is made of a probe which is complementary to the target sequence, dually labeled with a dye and a quencher (e.g. Dabcyl) at each of its two ends. In its closed state, the signal of the dye is quenched by the quencher. When the complementary sequence hybridizes to the target DNA, the beacon opens up and a signal can be detected. A further example of labels capable of specifically binding to an analyte and thereby causing a change in signal is provided for SERRS in WO2005/019812. Therein SERRS beacons are described which are dually labeled probes with a different dye at each of its two ends. The second dye is specifically designed such that it is capable of immobilizing the oligonucleotide probe onto an appropriate metal surface. In the absence of target DNA, the beacon is immobilized in the “closed state” on the metal surface, resulting in the detection of a SERRS spectrum corresponding to both dyes. When the complementary sequence hybridizes to the target DNA, the beacon opens up and one of the dyes is removed from the surface. This causes the SERRS signals to change. Alternatively, use can be made of fluorophore-labeled oligonucleotide probes whereby the polarization of the fluorescence of the label increases upon binding to the target nucleic acid (Walker and Linn (1996) Clinical Chemistry. 42:1604-1608). In a further embodiment use is made of a SE(R)RS-active label, of which the maximum absorption frequency is shifted from a first to a second frequency on association of the label with the SE(R)RS-active metal surface based on changed absorption spectra of adsorbed dye molecules to metal particle surfaces (as described by Franzen et al. (2002) J. Phys. Chem. 106:6533-6540; Noginov et al. (2005) J. Opt. A: Pure Appl. Opt. 7:S219-S229). According to this embodiment, the analyte is associated with the SE(R)RS-active metal surface. Upon contacting the SE(R)RS-active label with the sample, the SE(R)RS-active label that is specifically bound to the analyte is thereby associated with the metal surface, and emits a different spectrum than the SE(R)RS-active label that remains unbound in the sample. For example, a SE(R)RS-active labeled oligonucleotide probe may undergo a shift in maximum absorption frequency upon hybridization to DNA fragments that are associated with silver nanoparticles, and can therefore be detected in its hybridized (bound) as well as in its non-hybridized (unbound) form within the same sample.

According to another embodiment of the methods of the present invention, detection of the bound and unbound fraction of the label requires a prior separation of these fractions. Separation of the bound and unbound label can be achieved by any process that removes either the unbound labels and/or the analyte-bound label from the sample to allow individual detection thereof. Exemplary separation techniques include sedimentation, precipitation, centrifugation, specific binding to a substrate, gel electrophoresis, including but not limited to isoelectric focusing and capillary electrophoresis; dielectrophoresis; sorting, including but not limited to fluorescence-activated sorting techniques; chromatography, including but not limited to HPLC, FPLC, size exclusion (gel filtration) chromatography, affinity chromatography, ion exchange chromatography, hydrophobic interaction chromatography, immunoaffinity chromatography, and reverse phase chromatography. A detailed discussion of separation techniques can be found in, among other places, Rapley; Sambrook et al.; Sambrook and Russell; Ausbel et al.; Molecular Probes Handbook; Pierce Applications Handbook; Capillary Electrophoresis: Theory and Practice, P. Grossman and J. Colburn, eds., Academic Press (1992); Wenz and Schroth, PCT International Publication No. WO 01/92579; M. Ladisch, Bioseparations Engineering: Principles, Practice, and Economics, John Wiley & Sons (2001); and Liebler, Introduction to Proteomics, Humana Press (2002).

According to one embodiment, separation of the bound and unbound label is achieved by binding of the analyte to a substrate. Typically this involves a “capture probe”, which is bound to the substrate and capable of specifically binding the antigen. Where the analyte is a nucleotide sequence, the capture probe is typically an oligonucleotide complementary to a region within the analyte. Where the label is also bound to a probe, care is taken that the analyte-specific probe and the capture probe are complementary to different sequences within the analyte. Alternatively, the analyte is provided with a tag, which allows separation of the analyte (and consequently of the analyte-bound label) from the sample. This can be achieved e.g. when the target nucleotide sequence (analyte) is amplified using a tagged primer, whereby the tag allows binding to a substrate. According to a specific embodiment a biotin tag is introduced into the amplified analyte using a primer with a biotin tag. In the biotin-streptavidin capturing method the biotinylated analyte is captured by binding of the biotin molecule to a streptavidin-coated substrate, such as beads or streptavidin-coated wells of a microtitre plate.

Alternatively, where the analyte is a protein or peptide, the capture probe can be an analyte-specific antibody bound to a substrate. The binding of the analyte (and consequently the analyte-bound label) to a substrate allows the physical separation of the bound and unbound label. For instance, where magnetic beads are used, these can be removed from the sample by applying a magnetic field. Alternatively, the analyte can be captured by binding to immobilized capture probes fixed to a microtiterplate, after which the supernatant comprising the non-bound label can be removed.

According to yet another embodiment of the invention, detection is based on SE(R)RS and the separation step of the bound and unbound label fractions makes use of the SE(R)RS-active surface and/or label. More particularly, according to this embodiment the SE(R)RS-active surface and/or label inherently functions as or is/are provided with a tag which can be subjected to a physical or chemical force. For example, the weight of a SE(R)RS-active metal nanoparticle may be used in separation techniques as described in US 2005/0130163. Alternatively, the SE(R)RS-active surface comprises a tag which is a magnetic material (e.g. magnetic particles in a SE(R)RS-active bead) and separation of bound and unbound label fraction is ensured by applying a magnetic field or introducing a magnetic object in the sample. According to one embodiment, the magnetic SE(R)RS-active surface bound to the analyte-specific SE(R)RS-active label is added to the sample in a predetermined amount, whereupon a fraction of the magnetic SE(R)RS surface/analyte-specific SE(R)RS-active label binds to the analyte in the sample. The analyte is bound to a substrate by way of a capture probe. The unbound fraction of SE(R)RS-active label can be removed using an electromagnet and can be released (by turning off the magnet) in a separate vial. In another embodiment of the invention, an analyte-specific SE(R)RS-active label bound to a magnetic SE(R)RS-active surface and a biotinylated probe are used as (e.g. forward and reverse) primers for PCR-mediated DNA amplification of the analyte. The PCR product is both SE(R)RS- and biotin-labeled. An electromagnet introduced into a microtiter plate well containing the PCR product is switched on to collect all magnetic probes from the sample (i.e. incorporated in PCR product and unbound magnetic surface bound to SE(R)RS-active label). The magnet is then removed from the sample and dipped into another well, which is streptavidin-coated. When the magnet is switched off, the PCR product, also containing the biotin tag, is captured by the streptavidin. The unbound magnetic SE(R)RS-active labels can be removed by again switching on the electromagnet and transferred to another vial for detection according to the invention.

According to yet another embodiment, of the methods of the present invention, detection of the bound and unbound fraction of the label is performed within the same sample, i.e. without the actual removal of either of the fractions of the sample, but making use of different detection zones within one sample. Thus, according to this embodiment, a separation or physical movement of the bound and/or unbound fraction is ensured within a reaction vessel. Such a physical movement within a reaction vessel can be ensured by the use of analyte-specific labeled probes or a SE(R)RS-active surface provided with a tag which can be subjected to a physical/chemical force, allowing the movement of the bound and/or unbound probes. Examples of such forces include magnetic forces, electrokinetic forces, etc. According to a specific embodiment the tag on the analyte-specific probe is a ferromagnetic particle which, when subjected to a magnetic force is capable of moving the probe in the direction of the magnetic force.

The present invention relates to improved methods for the detection and/or quantification of an analyte, more particularly an analyte in a sample. While the methods described herein will generally refer to ‘an analyte’ it is equally envisaged that the methods of the present invention can be applied where several analytes are being detected or quantified simultaneously, using different analyte-specific labels. Most particularly, use can be made of different analyte-specific labels which can be differentially detected using the same detection method, such as, but not limited to different fluorescent labels (such as, but not limited to fluorescein isothiocyanates (FITC); carboxyfluoresceins (such as tetramethylrhodamine (TMR); carboxy tetramethyl-rhodamine (TAMRA); carboxy-X-rhodamine (ROX): sulforhodamine 101 (Texas Red™)) Atto dyes (Sigma Aldrich); Fluorescent Red and Fluorescent Orange; phycoerythrin, phycocyanin, and Crypto-Fluor™ dyes), quantum dots, or SE(R)RS-active dyes. As each of the labels will be specific for a different analyte, it is possible to measure, for each label, both the bound and unbound fraction, to obtain the internal validation of detection according the present invention.

The present invention allows for the improvement of the accuracy and the reliability of the detection and or quantification of one or more analytes in a sample. The term “sample” is used in a broad sense herein and is intended to include a wide range of biological materials as well as compositions derived or extracted from such biological materials. The sample may be any suitable preparation in which the analyte is to be detected. The sample may comprise, for instance, a body tissue or fluid such as but not limited to blood (including plasma and platelet fractions), spinal fluid, mucus, sputum, saliva, semen, stool or urine or any fraction thereof. Exemplary samples include whole blood, red blood cells, white blood cells, buffy coat, hair, nails and cuticle material, swabs, including but not limited to buccal swabs, throat swabs, vaginal swabs, urethral swabs, cervical swabs, throat swabs, rectal swabs, lesion swabs, abcess swabs, nasopharyngeal swabs, and the like, lymphatic fluid, amniotic fluid, cerebrospinal fluid, peritoneal effusions, pleural effusions, fluid from cysts, synovial fluid, vitreous humor, aqueous humor, bursa fluid, eye washes, eye aspirates, plasma, serum, pulmonary lavage, lung aspirates, biopsy material of any tissue in the body. The skilled artisan will appreciate that lysates, extracts, or material obtained from any of the above exemplary biological samples are also considered as samples. Tissue culture cells, including explanted material, primary cells, secondary cell lines, and the like, as well as lysates, extracts, supernatants or materials obtained from any cells, tissues or organs, are also within the meaning of the term biological sample as used herein. Samples comprising microorganisms and viruses are also envisaged in the context of analyte detection using the methods of the invention. Materials obtained from forensic settings are also within the intended meaning of the term sample. Samples may also comprise foodstuffs and beverages, water suspected of contamination, etc. These lists are not intended to be exhaustive.

In a particular embodiment of the invention, the sample is pre-treated to facilitate the detection of the sample with the detection method. For instance, typically a pre-treatment of the sample resulting in a semi-isolation or isolation of the analyte or ensuring the amplification of the analyte is envisaged. Many methods and kits are available for pre-treating samples of various types.

The preparation or pre-treatment of the sample will be determined by the detection method. For instance, when detection using SE(R)RS is envisaged, the sample may be in any appropriate form such as a solid, a solution or suspension or a gas, suitably prepared to enable recordal of its SE(R)RS spectrum. The detection sample can be at any suitable pH.

According to a particular embodiment of the invention, the analyte is a nucleic acid, such as a sequence of genomic DNA or a nucleic acid from a pathogenic microorganism. A variety of methods are available for isolating nucleic acids from samples. Exemplary nucleic acid isolation techniques include (1) organic extraction followed by ethanol precipitation, e.g. using a phenol/chloroform organic reagent (e.g. Ausbel et al., eds., Current Protocols in Molecular Biology, John Wiley & Sons, New York (1995, including supplements through June 2003), preferably using an automated DNA extractor, e.g., the Model 341 DNA Extractor available from Applied Biosystems (Foster City, Calif.); (2) stationary phase adsorption methods (e.g. U.S. Pat. No. 5,234,809; Walsh et al., BioTechniques 10(4): 506-513 (1991); and (3) salt-induced DNA precipitation methods (e.g. Miller et al., Nucl. Acids Res., 16(3): 9-10 (1988)), such precipitation methods being typically referred to as “salting-out” methods. Commercially available kits can be used to expedite such methods, for example, Genomic DNA Purification Kit and the Total RNA Isolation System (both available from Promega, Madison, Wis.). Further, such methods have been automated or semi-automated using, for example, the ABI PRISM™ 6700 Automated Nucleic Acid Workstation (Applied Biosystems, Foster City, Calif.) or the ABI PRISM™ 6100 Nucleic Acid PrepStation and associated protocols, e.g., NucPrep™ Chemistry: Isolation of Genomic DNA from Animal and Plant Tissue, Applied Biosystems Protocol 4333959 Rev. A (2002), Isolation of Total RNA from Cultured Cells, Applied Biosystems Protocol 4330254 Rev. A (2002); and ABI PRISM™ Cell Lysis Control Kit, Applied Biosystems Protocol 4316607 Rev. C (2001).

The above isolation methods can further comprise an enzyme digestion step, e.g. digestion with a proteolytic enzyme and/or an enzymatic amplification step, e.g. by PCR, and/or a shearing/sonication step for fragmentation.

As indicated above, the methods of the present invention are of particular interest in detection and/or quantification methods based on surface enhanced (resonance) Raman spectroscopy (SE(R)RS). Though reference is generally made to SE(R)RS herein, it will be understood that detection methods based on other types of spectroscopies are also envisaged, for example but not limited to surface enhanced fluorescence, normal Raman scattering, resonance Raman scattering, coherent anti-Stokes Raman spectroscopy (CARS), stimulated Raman scattering, inverse Raman spectroscopy, stimulated gain Raman spectroscopy, hyper-Raman scattering, molecular optical laser examiner (MOLE) or Raman microprobe or Raman microscopy or confocal Raman microspectrometry, three-dimensional or scanning Raman, Raman saturation spectroscopy, time resolved resonance Raman, Raman decoupling spectroscopy or UV-Raman microscopy.

In a particular embodiment of the invention, the method of the invention involves SERRS, since operating at the resonant frequency of the label gives increased sensitivity. In this case, the light source used to generate the Raman spectrum is a coherent light source, e.g. a laser, tuned substantially to the maximum absorption frequency of the label being used. This frequency may shift slightly on association of the label with the SE(R)RS-active surface and the analyte and/or analyte binding species, but the skilled person will be well able to tune the light source to accommodate this. The light source may be tuned to a frequency near to the label's absorption maximum, or to a frequency at or near that of a secondary peak in the label's absorption spectrum. SERRS may alternatively involve operating at the resonant frequency of the plasmons on the active surface.

In the methods of the invention based on SE(R)RS detection, typically one peak, corresponding e.g. to the label's absorption maximum, is selected for excitation and detection can be performed at a single wavelength of the “fingerprint” spectrum. Alternatively, especially when different analytes are being detected at the same time using different SERRS labels, the entire “fingerprint” spectrum may be detected in order to identify each label. However, also with different labels each having unique spectral lines the signal intensity may be detected at a chosen spectral line frequency or frequencies.

Typically, the detection step in a SE(R)RS based detection method will be carried out using incident light from a laser, having a frequency in the visible spectrum. The exact frequency chosen will depend on the label, surface and analyte. Frequencies in the red area of the visible spectrum tend, on the whole, to give rise to better surface enhancement effects. However, it is possible to envisage situations in which other frequencies, for instance in the ultraviolet or the near-infrared ranges, might be used. The selection and, if necessary, tuning of an appropriate light source, with an appropriate frequency and power, will be well within the capabilities of one of ordinary skill in the art, particularly on referring to the available SE(R)RS literature.

Excitation sources for use in SE(R)RS-based detection methods include, but are not limited to, nitrogen lasers, helium-cadmium lasers, argon ion lasers, krypton ion lasers, etc. Multiple lasers can provide a wide choice of excitation lines, which is critical for resonance Raman spectroscopy. According to a specific embodiment, an argon ion laser is used in a LabRam integrated instrument (Jobin Yvon) at an excitation of 514.5 nm.

The excitation beam may be focused on a substrate using an objective lens. The objective lens may be used to both excite the sample and to collect the Raman signal, by using a holographic beam splitter to produce a right-angle geometry for the excitation beam and the emitted Raman signal. The intensity of the Raman signals needs to be measured against an intense background from the excitation beam. The background is primarily Rayleigh scattered light and specular reflection, which can be selectively removed with high efficiency optical filters. For example, a holographic notch filter may be used to reduce Rayleigh scattered radiation.

The surface-enhanced Raman emission signal may be detected by a Raman detector. A variety of detection units of potential use in Raman spectroscopy are known in the art and any known Raman detection unit may be used. An example of a Raman detection unit is disclosed e.g. in U.S. Pat. No. 6,002,471. Other types of detectors may be used, such as a charge coupled device (CCD), with a red-enhanced intensified charge-coupled device (RE-ICCD), a silicon photodiode, or photomultiplier tubes arranged either singly or in series for cascade amplification of the signal. Photon counting electronics can be used for sensitive detection. The choice of detector will largely depend on the sensitivity of detection required to carry out a particular assay. Several devices are suitable for collecting SE(R)RS signals, including wavelength selective mirrors, holographic optical elements for scattered light detection and fibre-optic waveguides.

The apparatus for obtaining and/or analyzing a SE(R)RS spectrum may include some form of data processor such as a computer. Once the SE(R)RS signal has been captured by an appropriate detector, its frequency and intensity data will typically be passed to a computer for analysis. Either the fingerprint Raman spectrum will be compared to reference spectra for identification of the detected Raman active compound or the signal intensity at the measured frequencies will be used to calculate the amount of Raman active compound detected.

The present invention provides for improved methods for label-based detection of an analyte. Systems, kits, reagents and tools are within the scope of the present invention that are adapted to the application of the methods of the present invention, such as specifically adapted substrates (comprising areas with and without the capture probe) etc.

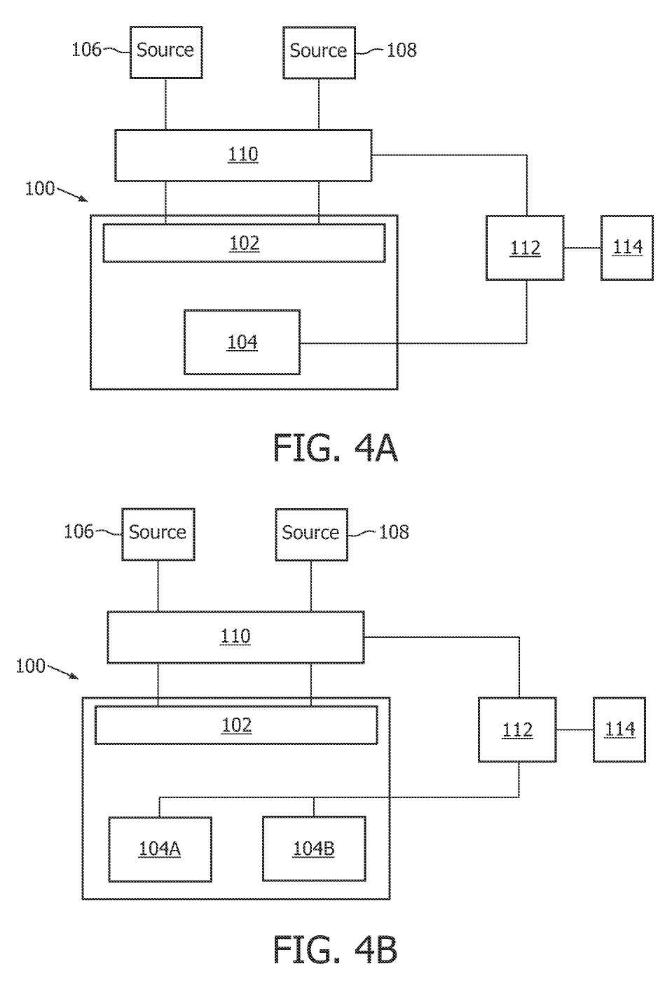

FIG. 4A is a schematic representation of the system according to an embodiment of the present invention. System (100) for detecting and optionally quantifying the presence of an analyte in a sample comprises source 106 for a sample suspected of containing an analyte and source 108 containing label capable of binding to the analyte, and means 110 for providing analyte and a predetermined amount of label to means 102 for contacting the sample comprising the analyte with the predetermined amount of label capable of binding to the analyte. The means 110 may include gravimetric feeds of the sample and/or analyte and may also include an arrangement of pipes/conduits and valves, e.g. selectable and controllable valves, to allow the provision of the fluids from sources 106, 108 to the contacting means 102. Alternatively, the fluids may be pumped from the sources 106, 108 to the contacting means 102.

The contacting means 102 may include means for separating the bound labels from the unbound labels according to any of the methods described above.

Control and analysis curcuitry 112 may be provided to control the operation of the means 110. Further, means 104 for detecting the fraction of label bound to the analyte and the fraction of label not bound to the analyte is also provided. The means 104 may be under the control of the analysis curcuitry 112. Signals representative of the detections may be supplied to the control and analysis curcuitry 112 which can be adapted to carry out algorithms to verify that the detections of unbound and bound labels are consistent with each other and to display the results on any suitable display means 114 such as a visual display unit, plotter, printer. The control and analysis curcuitry 112 may have a connection to a local area or wide area network for transmission of the results to a remote location. Control and analysis curcuitry 112 may be implemented in any suitable manner, e.g. dedicated hardware or a suitably programmed computer, microcontroler or embedded processor such as a microprocessor, programmable gate array such as a PAL, PLA or FPGA, or similar.

FIG. 4B shows an alternative embodiment of a system according to the present invention. Items with the same reference numbers as in FIG. 4A have the same function. The main difference between FIG. 4A and FIG. 4B is that the detection means 104 is provided as two different detecting means 104A and 104B for detecting of the unbound and bound labels, respectively. Any of the detection methods described above may be implemented by the detection means 104A and/or B.

### EXAMPLES

### Example 1

### Incorporation of an Internal Reference in the Detection of HIV

In the present example, detection of HIV is performed based on the presence of the gag gene (analyte) in a sample (as described by Isola et al., 1998, Anal. Chem. 70:1352-1356)

As a label cresyl fast violet (CFV) is used, which is incorporated into the analyte during a PCR amplification using a gag-specific oligonucleotide primer, which has been labeled with CFV (as described in Isola et al., above).

According to the present invention, a predetermined amount of CFV-labeled gag-specific oligonucleotide is used in the PCR reaction.

A capture probe is designed, which is a nucleotide sequence specific to a sequence of the gag gene within the sequence of the amplified PCR product, but different from the sequences of the PCR primers, which is provided with a linker (e.g. a six-carbon 5′-amino linker) capable of binding to a solid support such as a derivatized polystyrene plate. The capture probe is then spotted onto the support.

After blocking the unreacted sites on the derivatized plate, the double stranded PCR product within the PCR reaction mixture is denatured by boiling in water for 5 minutes and rapidly chilling on ice to prevent DNA reassociation. The mixture is added to the plate with the capture probe and allowed to hybridize in the presence of a hybridization solution. After hybridization, the buffer on the plate is carefully removed and transferred to another plate. The hybridization plate is rinsed with 100 μl buffer and the rinsing liquid is also collected.

The SERS-active surface is added after the hybridization by adding a 100 Å layer of silver by evaporation to both the hybridized plate and the plate containing the excess hybridization solution and rinsing liquid.

SERS spectra are taken from the two samples. The spectrum of the hybridized plate provides a direct indication of the amount of gag DNA in the sample. The spectrum of the excess hybridization solution comprising the fraction of unbound CFV-labeled gag-specific oligonucleotide, when deducted from the predetermined amount of CFV-labeled gag-specific oligonucleotide added to the sample, provides an internal control for the amount of gag DNA in the sample.

### Example 2

### Incorporation of an Internal Reference in the Detection of Chlamydia

In the present example, detection of the pathogenic bacterium Chlamydia trachomatis is performed based on the presence of the omp1 gene sequence (analyte) in a sample.

The omp1 gene in a sample is amplified by PCR using a forward primer tagged at the 5′-terminus with biotin, and a reverse primer in a first well.

A 17-base omp1-specific DNA oligonucleotide is tagged at the 5′-terminus with a substituted fluorescein dye, 2,5,1′,3′,7′,9′-hexachloro-5-carboxyfluorescein, available commercially as “HEX”. The resultant HEX-labeled omp1-specific oligonucleotide (“HEX probe”) has a sequence within the sequence of the amplified omp1 PCR product but different from the sequences of the PCR primers (“nested”), and is complementary to the strand in which the biotinylated primer is incorporated.

A predetermined amount of HEX probe is used for hybridization to the omp1 amplified biotinylated PCR product in the first well.

The biotinylated-hybridized complex is captured using streptavidin-coated magnetic beads. An electromagnet is switched on to collect all magnetic beads. The electromagnet is then removed from the solution and dipped into a second well for detection. The electromagnet is switched off to release all magnetic beads in the second well. In this way, all unbound HEX probes remain in the first well and all omp1-bound HEX probes are transferred to the second well. The latter are released from the biotinylated-hybridized complex by heat prior to detection. The excess biotinylated primers of the PCR reaction also bind to the streptavidin-coated beads are not HEX labeled and thus do not have a specifically detectable SERRS signal.

Detection of the unbound HEX probes in the first well and the heat-released HEX probes in the second well is performed as follows. Citrate reduced silver colloids are prepared according to the procedure described in U.S. Pat. No. 6,127,120. A solution of this colloid is prepared in distilled water. An aqueous solution of spermine hydrochloride is added to both wells, followed by an aliquot of the silver colloid solution. Spermine will ensure the formation of aggregated colloids thereby contributing to SERRS enhancement and will also aid in the adsorption of HEX probe onto the silver colloids. Both colloidal suspensions are subjected to SERRS examination.

The spectrum of the second well containing the fraction of bound probes i.e. the HEX probes specifically bound to omp1 prior to heat release, provides a direct indication of the amount of omp1 DNA in the sample. The spectrum of the first well containing the fraction of unbound probes when deducted from the predetermined amount of HEX probe added to the sample, provides an internal control for the amount of omp1 DNA in the sample.

### Example 3

### Incorporation of an Internal Reference in the Detection of a Predisposing Genetic Mutation

The DNA extracted from a patient sample is amplified using allele-specific oligonucleotides. Two forward primers are used along with one reverse primer. The forward primers are immobilized via a 5′-terminus linker onto SERRS-active beads comprising a SERRS-active label and a SERRS-active surface as described in US 2005/0130163. A predetermined amount of forward primer is used. The reverse primer is immobilized via the 5′-terminus with biotin. The PCR product incorporates both the SERRS-active bead and the biotin tag.

The mixture of the PCR reaction is spotted onto a streptavidin-coated microtiter plate. Both the biotinylated PCR product and the biotinylated primers are captured on the plate. The excess fluid is removed from the plate and transferred to a non-coated plate. This contains the excess forward primer linked to the SERRS-active beads.

SERRS spectra are taken on the immobilized SERRS-active beads and the unbound SERRS-active beads.

The ability of SERRS to identify different labels without separation makes it possible to use different primers with different labels and identify the presence of PCR product (and unbound forward primer) for each of the primers in one sample.

### Example 4

### Incorporation of an Internal Reference in the Detection of Eubacterial DNA

Bacteria are frequently found as contaminants in cell cultures. Studies have identified an overall 6.5% incidence of static bacterial contamination of cell cultures examined. Thus, many cell cultures lack visual signs of bacterial contamination, generally indicated by decoloration of the fluid. Moreover, it has been demonstrated that standard antibiotics not only are uneffective against resistant bacterial infection but also have a strong impact on the metabolism, cell growth and differentiation.

Using a probe specific to the 16S ribosomal RNA coding region in the eubacteriae genome, it is possible to detect the most common eubacteria species usually encountered as airborne contaminants in cell cultures.

The sensitivity of SERRS detection makes it possible to detect very low concentrations of DNA, and thus to forego the amplification step.

A sample of cell supernatant is contacted with a predetermined amount of the 16S RNA-specific probe, linked to a Cy3 SERRS label, and allowed to hybridize.

The hybridization mixture is spotted onto a plate to which a different 16S RNA-specific probe has been linked. After allowing the hybridization of the target DNA to which the Cy3 label is bound to the capture probe, the fluid is removed and transferred to a second plate. The plate is rinsed and the rinsing fluid is also transferred to the second plate.

The SE(R)RS-active surface is added to the first plate after the hybridization by adding a 100 Å layer of silver by evaporation. The presence of unbound 16S RNA specific probe in the second plate is determined by fluorescence.

### Example 5

### Incorporation of an Internal Reference in the Detection of hGH in a Sandwich ELISA

Silver electrodes are incubated at 37° C. and are then incubated in a solution of anti-human Growth Hormone (hGH) in 1% NaHCO as described in U.S. Pat. No. 5,266,498. The electrodes are then saturated with a BSA solution.

A dilution range of a sample comprising hGH and of a standard of hGH are made in buffer and the silver films are incubated with the different concentration batches of sample and standard. After being washed, the films are contacted with a predetermined amount of diaminobenzidine (DAB)-labeled anti-hGH (e.g. 40 μg/ml) and incubated. The reaction fluid is removed and transferred to a detection vial. The films are further rinsed.

SERRS spectra are obtained of the electrodes. Concentration of the unbound DAB-labeled anti-hGH in the reaction fluid is determined enzymatically, based on comparison with a standard.

The values obtained by the direct detection of the DAB-labeled anti-hGH bound to the silver films and by the detection of the unbound DAB-labeled anti-hGH are compared to determine the reliability of the detection.

1. A method for detecting and/or quantifying an analyte in a sample, comprising the steps of:
a) contacting said sample comprising said analyte with a predetermined amount of label capable of binding to said analyte;
b) detecting the fraction of label bound to said analyte; whereby the amount of label bound to said analyte is indicative of the presence and/or amount of said analyte in said sample; and
c) detecting the fraction of label not bound to said analyte; whereby the amount of label not bound to said analyte, deducted from said predetermined amount of label, provides an internal control indicative of the presence and/or amount of said analyte in said sample.
wherein said detection step in (b) and (c) is ensured using an optical detection method.

2. The method of claim 1, wherein said detection step (b) and said detection step (c) is performed within the same sample.

3. The method according to claim 1, wherein said detection step in (b) and (c) is ensured using SE(R)RS and wherein said label is a SE(R)RS-active label.

4. The method according to claim 3, further comprising, prior to step (b) and (c), contacting of said fraction of label bound to said analyte and of said fraction of label not bound to said analyte with a SE(R)RS-active surface.

5. The method according to claim 1, wherein said label is an analyte-specific label.

6. The method according to claim 1, wherein said analyte-specific label comprises an analyte-specific probe.

7. The method according to claim 1, wherein said analyte is a nucleotide sequence and said analyte-specific probe is a oligonucleotide having a sequence complementary to a sequence within said analyte.

8. The method according to claim 2, wherein use is made of a label which allows differential detection of said label bound to said analyte and said label which is not bound to said analyte.

9. The method according to claim 1, wherein said label is a fluorescent and/or a SE(R)RS-active label of which the maximum absorption frequency is shifted from a first to a second frequency on association of said fluorescent and/or SE(R)RS-active label with said SE(R)RS-active surface.

10. The method of claim 1, further comprising, prior to step (b) the step of separating the fraction of label bound to said analyte from the fraction of label not bound to said analyte.

11. The method according to claim 10, wherein said separation of said fraction of bound label and said fraction of unbound label is ensured by making use of an analyte-specific probe which is provided with a tag which can be subjected to a physical or chemical force.

12. The method according to claim 10, wherein said label is a SE(R)RS-active label, wherein prior to step (b) and (c) said fraction of label bound to said analyte and said fraction of label not bound to said analyte are contacted with a SE(R)RS-active surface and wherein said separation is ensured by using a SE(R)RS-active surface which functions as or is provided with a tag which can be subjected to a physical or chemical force.

13. A system for detecting and/or quantifying the presence of an analyte in a sample, comprising:
a) means for contacting said sample potentially comprising said analyte with a predetermined amount of label capable of binding to said analyte;
b) means for detecting the fraction of label bound to said analyte; whereby the amount of label bound to said analyte is indicative of the presence and optionally of the amount of analyte in said sample; and
c) means for detecting the fraction of label not bound to said analyte; whereby the amount of label not bound to said analyte, deducted from said predetermined amount of label, provides an internal control indicative of the presence and optionally the amount of analyte in said sample,
wherein said detection is ensured using an optical detection method.
